(19) United States
(12) Patent Application Publication
(10) Pub. No.: US 2009/0075781 A1
(43) Pub. Date: Mar. 19, 2009

(54) SYSTEM FOR INCORPORATING DATA FROM BIOMETRIC DEVICES INTO A FEEDBACK MESSAGE TO A MOBILE DEVICE

(75) Inventors: Robert Schwarzberg, Boca Raton, FL (US); Marion Zabinski, San Diego, CA (US); Rene Melton, Delray Beach, FL (US); Timothy J. Dion, Parkland, FL (US)

Correspondence Address: STANDLEY LAW GROUP LLP, 6300 Riverside Drive, Dublin, OH, 43017, US

(73) Assignee: Sensei, Inc., Boca Raton, FL (US)

(21) Appl. No.: 12/118,144

(22) Filed: May 9, 2008

Related U.S. Application Data
(63) Continuation-in-part of application No. 12/117,190, filed on May 8, 2008.
Application No. 12/117,190 is a continuation-in-part of application No. 11/856,917, filed on Sep. 18, 2007.

(51) Int. Cl. A61B 5/00

(52) U.S. Cl. 482/8

(57) ABSTRACT

A system and method for using biometric data received from a user to generate feedback messages for transmission to the user while the user performs a physical activity. Wireless biometric sensors such as those used to monitor heart rate, blood pressure, and pulse rate are integrated into a user's diet and exercise plan and the personalized instruction that the individual receives. An expert system uses the biometric data to monitor the effectiveness of the physical activity and generate feedback messages. The expert system then sends the messages to the user's mobile device. The messages may inform the user of the biometric data, may provide positive reinforcement related to the user's progress toward a stated goal, or may suggest modifications to the physical activity to assist the user in reaching a stated goal.

### CROSS-REFERENCE TO RELATED APPLICATIONS

This application is a continuation-in-part application of U.S. patent application Ser. No. 12/117,190, filed May 8, 2008, titled METHOD FOR TAILORED STRATEGY MESSAGES FROM AN EXPERT SYSTEM TO ENHANCE SUCCESS WITH MODIFICATIONS TO HEALTH BEHAVIORS, which is incorporated herein by reference and is a continuation-in-part application of U.S. patent application Ser. No. 11/856,917 filed Sep. 8, 2007, titled SYSTEM AND METHOD FOR REWARDING USERS FOR CHANGES IN HEALTH BEHAVIORS, which is incorporated herein by reference.

### TECHNICAL FIELD

The present invention relates generally to systems and methods for assisting with the maintenance of healthy lifestyle habits. More particularly, the present invention is a system and method for integrating wireless biometric sensors and related data into a diet and exercise plan.

### BACKGROUND OF THE INVENTION

Dieting has become an extremely popular activity resulting from people's awareness of the health risks of becoming overweight or obese, a desire to improve one's appearance, and an aspiration to achieve the sense of accomplishment that comes from setting a difficult goal and accomplishing it. However, there is no singular method of dieting that works for every person. Body types, weight loss goals, and preferences vary greatly depending on the individual. Every dieter has individual likes and dislikes as to types of food, times and places to eat, type and length of exercise, eating habits, etc. Due to these differences, many dieters become frustrated with rigid, impersonal diets, and often quit the diet after a short time.

Furthermore, dieters differ on how well they can motivate themselves to continue to adhere to certain dietary guidelines. For example, a dieter who is supposed to only eat a cup of pasta and a vegetable for lunch, but instead decides to eat an ice cream cone as well may be unable to justify such a decision within the diet and decide to give up the diet for the rest of the day. Because such “splurges” are detrimental to the dieter's physical and mental progress, the dieter may find the diet unsustainable.

Many diet plans fail to allow for individualized exercise schedules. Typically, the diet plans suggest the same workout schedule for every person on the diet. For example, the diet plan may incorporate a workout of a half hour, two to three times a week. Such a generalized workout schedule has a number of weaknesses. First, it fails to factor in the type of activity the person is performing. The effectiveness of the exercise depends on what exercise is performed as well as the intensity. A half hour of strolling through a park is simply not as effective as a half hour of running.

Second, it does not allow for individualized ability. For some people, working out for half an hour, two to three times a week, may be physically impossible given their current condition. It may be possible for them in the future, but the person may be limited to a 15 minute workout for until his or her health improves. Other individuals may have the desire and ability to workout for a longer period of time or a greater number of times per week.

Another problem with a general workout schedule is that it fails to factor in the individual's likes and dislikes as to type of activity and time of day to perform the activity. A plan that proposes exercise two to three times a week is easily put aside when the person is busy and distracted by other activities. A person is much more likely to perform an activity that is scheduled or better yet, for which a reminder is provided telling the person to perform a certain activity at a certain time.

Finally, existing diet plans often fail to take into account details regarding the user's dietary information. The two parts of the diet plan—the intake of food and exercise—are generally treated as separate parts of the plan. However, the two are related in that they both have an impact on caloric intake. The more one exercises, the more one can eat without gaining weight. Accounting for both activities allows the impact of one to be applied to the other. For example, a person may eat a large lunch one day and reduce the impact of the lunch by including an extra work out later in the week. Likewise, a person could miss a scheduled workout and compensate for it by eating a smaller meal later in the day.

A highly effective solution to many diet program problems is found through the use of coaching. Research has shown that dieting and weight loss is more successful when the dieter is coached throughout the process. Coaching keeps the dieter motivated, provides positive reinforcement, and introduces a narrowly-tailored plan for each individual participant. However, obtaining a reliable human coach is difficult and prohibitively expensive such that relatively few dieters are actually able to use one.

### SUMMARY OF THE INVENTIVE CONCEPT

The present invention addresses the diet plan problems identified above by providing personalized plans to meet the needs and requirements of individuals. It uses tailored messages to “coach” individuals in following their personalized plans. A system and method for integrating wireless biometric sensors and related data into a personalized diet and exercise plan helps an individual meet a specified goal. An expert system uses information about an individual's diet and exercise preferences as well as biometric data to provide tailored messages related to the plan. The individual receives personalized instruction in the dieting field, without having to pay the prohibitively expensive fees that are typically associated with personalized instruction. Exemplary embodiments allow the individual to use portable devices and technology, such as cell phones, PDA's, Blackberrys™, iPhones™, and others, so that the individual has constant access to personalized instruction regarding his or her personalized diet and exercise plan.

The personalized instruction is based on an individual's diet plan preferences, food preferences, meal preparation preferences, and exercise preferences. Once the dieter's plan has been established, typically by providing preference and other information through an online website, the dieter is never required to access the site again as the personalized instruction is given through the dieter's portable technology. The prior art is known to center around “pull” technology, where the user must reach out to the system for the information, and if there is inaction by the user, the instruction will stop. Use of “push” technology—where the instruction is sent to the user—and inaction by the user prompt the systems to reach out to the user provides for encouragement and corrective actions.

It has been found, through the study of behavior informatics, that the use of technology can help people make significant changes in their health. Gradual change, over a longer period of time, is more effective for long-term health solutions, rather than behavior changes that are expected to take place rapidly, over a short period of time. Further, many dieters are more comfortable using familiar technology to assist them with their dieting, as opposed to unfamiliar and possibly uncomfortable office and training room sessions with an actual dietitian and trainer. These concepts are incorporated into a diet and exercise instructional platform based on individuals' preferences to increase the likelihood that individuals will adopt and follow a plan that helps them reach their personal goals.

Wireless biometric sensors such as those used to monitor heart rate, blood pressure, pulse rate, pedometer activities, and other parameters, are integrated into an individual's diet and exercise plan and the personalized instruction that the individual receives. These devices allow an expert system to monitor the effectiveness of the exercise the user has accomplished. The expert system then sends out messages to the wireless communications device (e.g., mobile phone) to be presented to the user. The messages may inform the user of his/her biometric data, may provide positive reinforcement related to the individual's progress, or may make suggestions on how the individual can change his or her performance to better meet a goal. Furthermore, the individual may send a message in response that is used by the expert system to determine the need for additional feedback.

Message provided by the expert system vary in complexity. Some messages may simply forward the individual's heart rate or distance traveled during a jogging session. The process may also consider a number of individualized characteristics, such as the person's body type, the person's weight loss goals, the amount of time the person has to spend on the activity, the amount and type of food the person has eaten or will eat, the amount and type of prior or future workout sessions, etc. These factors can be considered in relation to data that demonstrates the effectiveness of certain exercises and exercise rates on people with similar characteristics. The messages may be designed to help increase the individual's activity level and stamina. The expert system may store information about the completed activity and integrate it with other data as established in a profile. In this manner, the exercise schedule as well as the dietary schedule can be dynamically updated and changed accordingly.

Furthermore, embodiments may also include the use of the wireless sensors even when a scheduled activity is not being performed. The sensors may be used to monitor the amount of exercise that results during the day and is incidental to the individual's normal, daily activities. This data can be added to the individual's profile and used to develop a more individualized exercise and dietary schedule.

In addition to the novel features and advantages mentioned above, other features and advantages will be readily apparent from the following descriptions of the drawings and exemplary embodiments.

### BRIEF DESCRIPTION OF THE DRAWINGS

FIG. 1 illustrates the physical structure of a system according to an example embodiment.

FIG. 2 is a screen shot of one embodiment of the exercise plan interface.

FIG. 3 is a screen shot of one embodiment of the daily schedule interface.

### DETAILED DESCRIPTION

Exemplary embodiments provide a system for displaying messages on a mobile wireless device based on the data read from a biometrics device associated with a user. In further embodiments, the message is tailored toward the user's personal weight loss and exercise program.

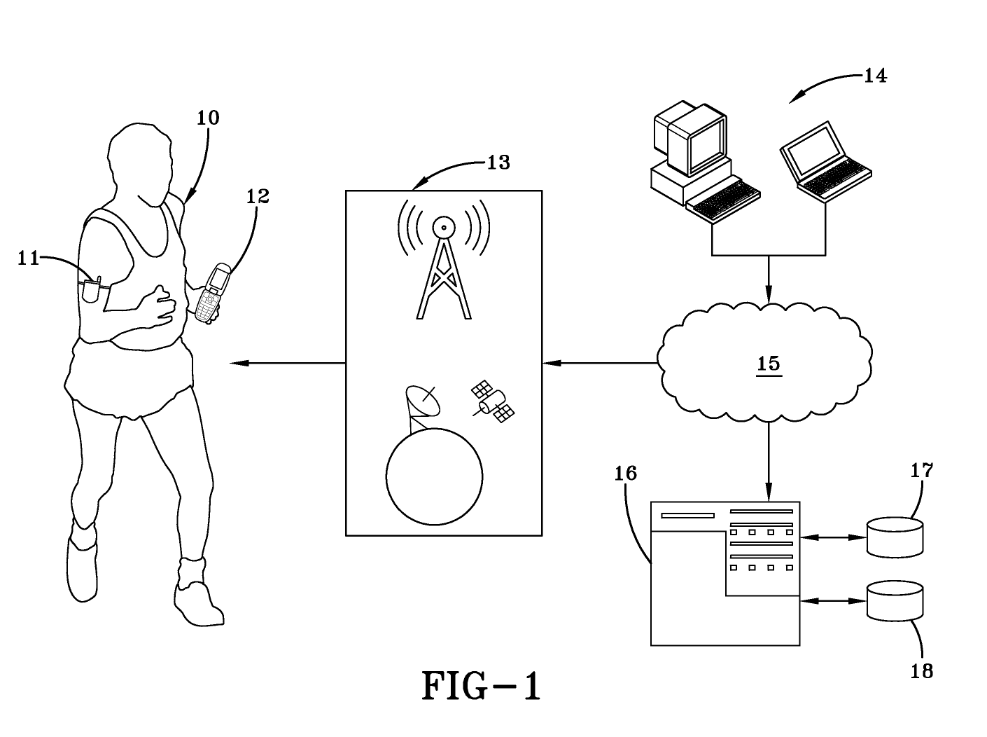

FIG. 1 is an illustration of the physical structure of a system according to an example embodiment. Connections between components permit data to flow in both directions. A laptop or desktop personal computer 14 is connected to a server 16 through the internet 15. The server 16 is connected to one or more databases 17, 18 comprising user data, nutritional and recipe, diet, and exercise data, message data, and other data as may be required to provide the features and functionality. The server 16 is connected to communication networks 13 through the internet 15. The various communication networks 13 facilitate communications with a user 10 portable technology 12 which may include cellular or mobile phones, personal digital assistants, or any other portable device capable of sending and receiving communications through the communication networks 13.

A biometric device 11 may be associated with the user such that it reads the user's biometric data and transmits it to the communications network 13, which relays the signal to the server 14 for use by an expert system. The biometric device 11 may monitor heart rate, blood pressure, pulse rate, pedometer activities, and other parameters. Based on the biometric data received by the expert system, the expert system stores and analyzes the data and sends a message to be displayed on a mobile device 12. The message is relayed through the communications network 13 and sent to the mobile device 12. The mobile device 12 may consist of any one of the following: cell phones, PDA's, Blackberrys™, iPhones™, any other portable device that is capable of receiving messages and displaying them for a user.

The biometric device 11 may be a conductive fiber, optical device, a pulse transducer or any other device known in the art for calculating biometrics. Conductive fibers involve fabrics that can conduct electricity for monitoring many different types of activities. Electrical conductivity is supplied through elastic, highly conductive, composite fibers that can be incorporated into apparel. Examples of activities that can be monitored through the use of conductive fibers include heart rate, blood pressure, pulse rate, and pedometer activities. Optical devices use chip based technology that allow for tracking of heart rate and pedometer activity through contact with the wrist. Pulse transducers are based on piezo technology, such that the pulse transducer converts force that is applied to the transducer surface into electrical signals, which can be monitored and analyzed. A user's heart rate can be monitored using pulse transducers.

In one embodiment, the biometric sensors have means to wirelessly communicate to the same remote, expert system as the wireless communications device. The expert system receives a message detailing the user's biometrics, stores the message, integrates the message with the user's personal diet and exercise profile, and sends out a reply message related to biometric data.

The analysis completed by the expert system at the server 14 to formulate the message may simply consist of forwarding the biometric data or it may consist of relating the data to the user's diet and exercise plan data. Plan data may include what the user has eaten, what the user will eat, what activities the user has performed and their effectiveness, what activities the user will perform and their projected effectiveness, the user's goals as to weight loss and exercise, and/or personal information about the user such as body type, weight, and height. Relating the biometric data to the user's diet and exercise plan data may involve determining how effective the activity is and comparing the data with the user's goal for the activity. Determining the goal or effectiveness of the activity may involve calculating how many calories are being burned, or the rate of calories burned per period of time, amount/rate of fat burned, etc. The amount of time the user has been performing the activity and/or how long the activity is expected to take may also be used in the calculations. The messages may make encouraging remarks about the individual's progress, or may make suggestions on how the individual should change his/her performance to better meet the goal of the activity. Examples include messages such as “Speed Up!,” “Slow down! You're going too fast!,” “Great Job! Keep it up!,” or “Try walking instead of jogging.” Furthermore, the individual may send a message to the expert system in response.

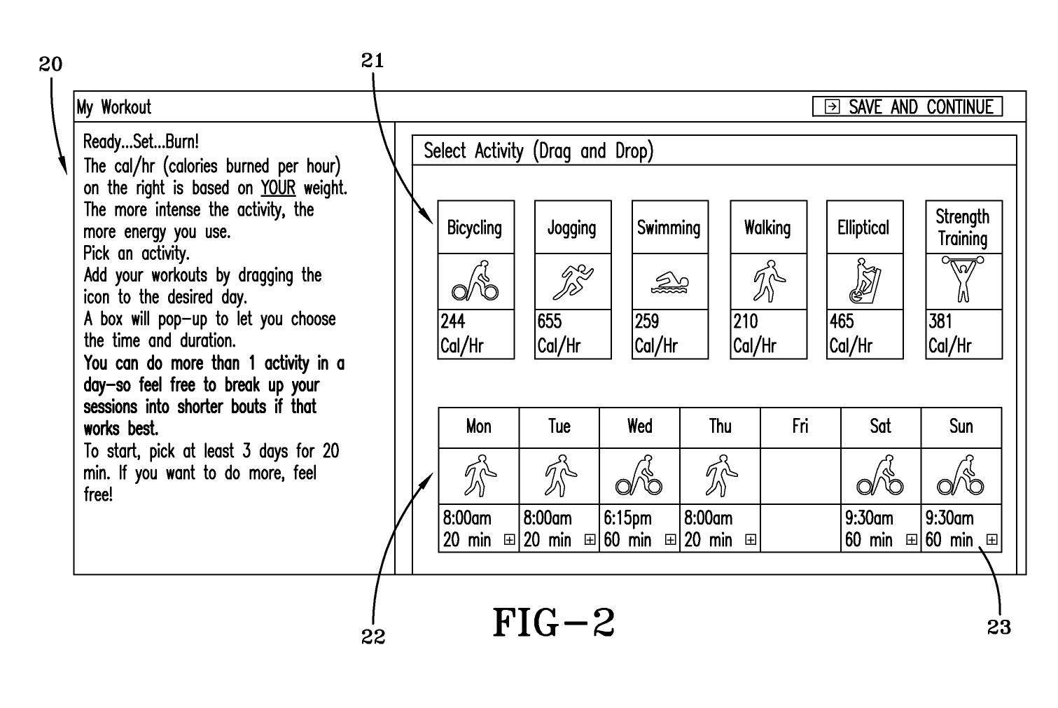

The expert system may also store the biometric data for later analysis. This analysis may include determining the overall effectiveness of the entire activity, integrating this data with diet and exercise plan data, and dynamically changing a diet and exercise schedule. The diet and exercise schedule comprises types of food to eat and exercise to perform and at what times both should be completed (e.g., as shown in FIG. 2, item 24). The diet and exercise schedule may be changed to account for the actual effectiveness of the exercise was compared to the projected effectiveness before it was completed. If the exercise was more effective than projected, extra food may be added to the schedule and/or exercise may be deleted from the schedule. If the exercise was less effective than projected, food may be deleted and/or exercise added to the schedule. The user may be queried to decide how to change the schedule or the expert system may automatically change the schedule.

In a further embodiment, the user 10 is not performing an activity, but is simply performing everyday tasks. In this embodiment, the expert system may sample and store the user's biometric data throughout the day for further analysis. The individual's overall daily activities may be considered in developing a weekly or monthly diet and exercise schedule.

Users complete an enrollment process in which they provide information for a profile comprising diet and exercise preferences and identifying health behavioral challenges. A user may provide contact and background information (such as information for contacting the user's cell phone), specify a sex, age, weight, height, and weight goal, specify preferences related to diet and exercise, and identify personal behavioral challenges. The user's profile data and specified goal and diet and exercise preferences are considered by the expert system to tailor messages. The user accesses a website to navigate through the screens and provide data and information that allows the system to build a profile for the user comprising diet and exercise preferences as well as behavioral challenges.

A user may specify a type of diet plan he or she would like to follow (e.g., a balanced plan that emphasizes a diet of reduced calories as well as reduced fat and sugar, a healthy carbohydrate plan that emphasizes a diet of lean meats, fish, dairy, and nuts, or a Mediterranean plan that emphasizes a diet of fish, grains, fruits, vegetables, beans, and nuts). The user also specifies food preferences by selecting a food category and identifying the foods in each category that he or she does not like or wants to avoid. The user may further specify a time of day for eating breakfast, lunch, and dinner as well as a snack. The user may specify a meal preparation preference that indicates the user's preferences for obtaining breakfast, lunch, and dinner on weekdays and weekends. Meal preparation options may include: 1) quick and easy (fewer than 10 minutes to prepare); 2) cook at home (more than 10 minutes to prepare); 3) frozen or ready to eat; 4) fast food; or 5) order from restaurant. The meal preparation preferences provide additional data for the expert system to consider when generating messages to the user related to meal suggestions. Finally, the user may identify behavior challenges that interfere with the user's ability to reach a particular health goal.

Referring to FIG. 2, a screen shot of an exercise plan interface according to an example embodiment is shown. There are instructions 20 informing the user of how to choose an exercise plan. There is a list of activities from which to choose 21, each with a picture icon of the activity and information on approximately how many calories/hour are burned while performing the activity. A selected activity is chosen from the list 21 and dragged to a selected day of the week on the schedule below 22. The user is then queried for a time of day to begin the selected activity and the amount of time to perform the activity 23. This information is saved and the expert system may use it to formulate a goal for the user and to develop a plan for the user. Another webpage may be used to input a user's goals as to weight loss, exercise stamina and rate, or exercise biometrics goals. A message may be sent to the mobile device 12 a few minutes before the activity is to be performed reminding the user to set up the biometric device 11 and complete the activity.

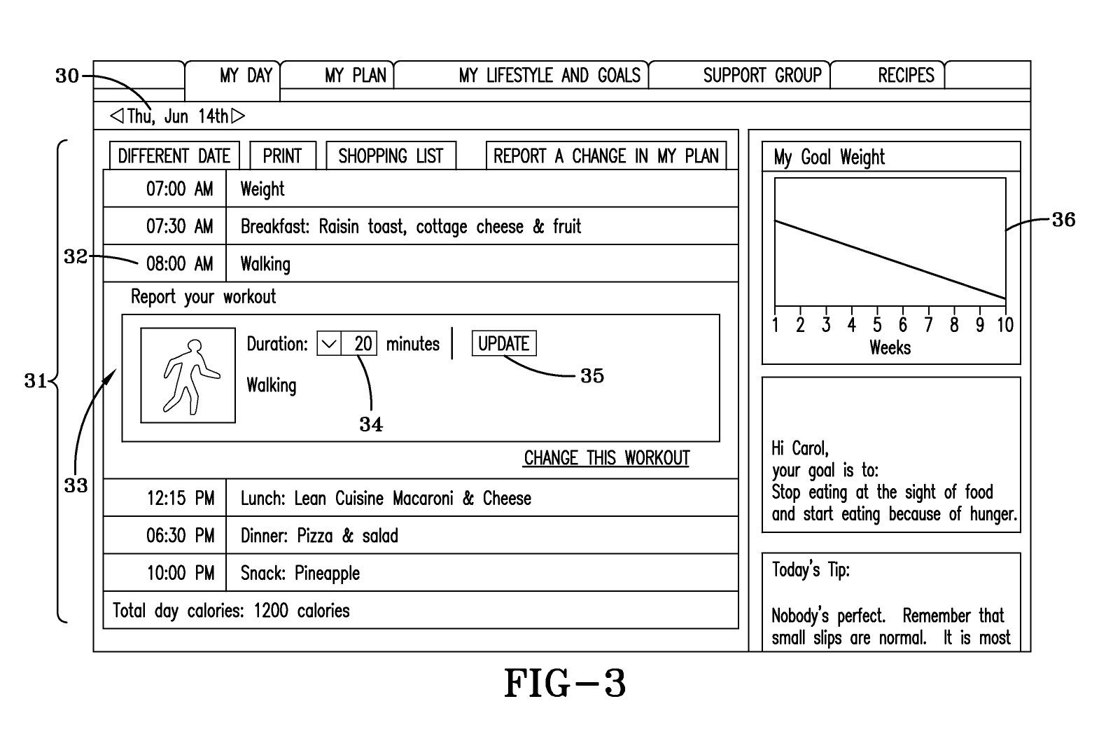

FIG. 3 is a screen shot of one embodiment of the diet and exercise schedule interface. It shows the date 30 which can be moved up or back as well as a complete meal schedule for the day 31 and a physical activity included 32. If the physical activity is selected, a more detailed explanation is shown in the box below 33, where the activity duration can be changed 34. Also, the entire schedule may be updated if the “update” option 35 is selected. A goal weight listing is also shown 37.

The expert system uses an individual's exercise plan and schedule information as well as biometric data that it receives to tailor messages regarding the individual's exercise program. As indicated previously, the expert system may provide feedback to an individual during a physical activity suggesting a modification to the intensity level of the activity to help the individual reach a weight or exercise goal. Alternatively, if the individual is exercising at an appropriate level of intensity, the expert system may simply encourage the individual to keep performing the activity until the activity is scheduled to end.

Any embodiment may include any of the optional or preferred features of the other embodiments. The exemplary embodiments herein disclosed are not intended to be exhaustive or to unnecessarily limit the scope of the invention. The exemplary embodiments were chosen and described in order to explain the principles so that others skilled in the art may practice the invention. Having shown and described exemplary embodiments, those skilled in the art will realize that many variations and modifications may be made to affect the described invention. Many of those variations and modifications will provide the same result and fall within the spirit of the claimed invention. It is the intention, therefore, to limit the invention only as indicated by the scope of the claims.

What is claimed is:

1. A method for incorporating biometric data of into feedback for an exercise program, comprising:
(a) receiving from a user exercise program data, said exercise program data comprising data for a physical activity for said user to perform and a goal related to performance of said physical activity;
(b) capturing biometric data from a biometric device used by said user while performing said physical activity;
(b) transmitting said biometric data from said biometric device to a remote server;
(c) generating at said server a feedback message for said user relating to said user's performance of said physical activity and based on said biometric data and said goal related to performance of said physical activity; and
(d) transmitting said feedback message from said server to a mobile device used by said user while performing said physical activity.

2. The method of claim 1, further comprising (e) storing said biometric data with a profile for said user, said profile comprising diet and exercise preference data for said user.

3. The method of claim 1, wherein generating said feedback message comprises generating a feedback message suggesting a modification to an intensity level of said physical activity to assist said user in reaching said goal related to performance of said physical activity.

4. The method of claim 3, wherein suggesting a modification to an intensity level comprises suggesting to said user to perform said physical activity at a higher rate.

5. The method of claim 1, wherein said goal is selected from the group consisting of burning a specific number of calories during said physical activity and performing said activity for a specific period of time.

6. The method of claim 1, wherein said biometric device is selected from the group consisting of a conductive fiber, optical device, and a pulse transducer.

7. The method of claim 1, wherein said mobile device is a cellular phone.

8. The method of claim 1 wherein said physical activity is an exercise.

9. The method of claim 1 wherein said physical activity is one incidental to said user's normal, daily activities.

10. A system for incorporating data from biometric devices into a feedback message to a user's mobile device, comprising:
at least one biometric device associated with a user for measuring biometric data while said user performs a physical activity;
a transmitter in communication with said biometric device for transmitting biometric data to a remote server;
an expert system at said server for generating a feedback message for said user relating to said user's performance of said physical activity and based on said biometric data and a goal for said user related to performance of said physical activity; and
a mobile device for receiving said feedback message from said expert system while said user is performing said activity.

11. The system of claim 10 wherein said goal is selected from the group consisting of burning a specific number of calories during said physical activity and performing said activity for a specific period of time.

12. The system of claim 11, wherein said goal is adjusted by said expert system based on said biometric data.

13. The system of claim 12, wherein said feedback message includes a suggestion from the group consisting of performing said physical activity at a higher rate, performing said physical activity at a lower rate, or no longer performing said physical activity.

14. The system of claim 12, wherein said mobile device is a cellular phone.

15. A method for incorporating biometric data of a user into a feedback message to a mobile device, comprising:
receiving at a server biometric data from at least one biometric device in use while said user performs a physical activity;
calculating at said server effectiveness data using said biometric data, a time allotted to perform said physical activity, and profile data for said user;
comparing said effectiveness data with a goal of said user related to said physical activity;
generating a feedback message for said user related to said effectiveness data for said physical activity; and
transmitting said feedback message to said mobile device.

16. The method of claim 15, wherein generating said feedback message comprises generating a feedback message suggesting a modification to an intensity level of said physical activity to increase effectiveness of said physical activity according to said effectiveness data.

17. The method of claim 16, wherein suggesting a modification to an intensity level comprises suggesting to said user to perform said physical activity at a rate selected from the group consisting of a higher rate and a lower rate.

18. The method of claim 1, wherein said goal is selected from the group consisting of burning a specific number of calories during said physical activity and performing said activity for a specific period of time.

19. The method of claim 1, wherein said biometric device is selected from the group consisting of a conductive fiber, optical device, and a pulse transducer.

20. The method of claim 1 wherein said physical activity is selected from the group consisting of an exercise and an activity incidental to said user's normal, daily activities.
